(19) United States
(12) Patent Application Publication
(10) Pub. No.: US 2016/0074013 A1
(43) Pub. Date: Mar. 17, 2016

(54) IMAGE ENLARGEMENT METHOD AND ULTRASOUND MEDICAL DEVICE FOR SAME

(71) Applicant: ALPINION MEDICAL SYSTEMS CO., LTD., Gyeonggi-do (KR)

(72) Inventors: Supyeong CHAE, Incheon (KR); Hyunchul CHO, Ansan-si (KR); Keonho SON, Seongnam-si (KR); Sun-Yeob CHANG, Seoul (KR)

(21) Appl. No.: 14/787,931

(22) PCT Filed: Apr. 30, 2013
(86) PCT No.: PCT/KR2013/003763

§ 371 Date: Oct. 29, 2015

(30) Foreign Application Priority Data
Apr. 30, 2013 (KR) 10-2013-0048827

(51) Int. Cl. A61B 8/08; A61B 8/14; A61B 8/00

(52) U.S. Cl. CPC A61B 8/5207; A61B 8/463; A61B 8/4483; A61B 8/145; A61B 8/469

(57) ABSTRACT

An image zoom method and an ultrasound medical apparatus are disclosed. An image zoom method that allows an entire image to be updated in real time in a zoom reference window in a write zoom system of two zoom systems (read zoom and write zoom) employed in an ultrasound medical apparatus, to increase diagnosis efficiency, and an ultrasound medical apparatus employing the image zoom method are provided.

### TECHNICAL FIELD

The present disclosure relates to an image zoom method and an ultrasound medical apparatus, and more particularly, to an image zoom method that allows an entire image to be updated in real time in a zoom reference window in a write zoom system of two zoom systems (read zoom and write zoom) employed in an ultrasound medical apparatus, to increase a diagnosis efficiency, and an ultrasound medical apparatus employing the image zoom method.

### BACKGROUND

The statements in this section merely provide background information related to some embodiments of the present disclosure and do not necessarily constitute prior art.

An ultrasound system has noninvasive and nondestructive characteristics, and hence it is widely used in a medical field to acquire internal information of a subject. The ultrasound system is widely used in the medical field as it provides a high-resolution image of the inside of the subject by using an ultrasound in real time instead of a surgical operation to directly incise and observe the subject. Such an ultrasound system transmits an ultrasound signal to the subject, receives a reflected signal from the subject to form an ultrasound image of the subject, and provides an image zoom function of magnifying the ultrasound image. That is, when a zoom area is set on the ultrasound image, the ultrasound system magnifies the image corresponding to the zoom area.

A general image zoom function only shows the zoom area of the subject to be diagnosed, which cannot provide the entire image of the subject in real time or the entire image with high resolution. This leaves a user with a difficulty of real-time checking of the entire image other than the zoom area.

### DISCLOSURE

### Technical Problem

The present disclosure has been made in view of the above aspects, and the present disclosure seeks to provide an image zoom method that allows an entire image to be updated in real time in a zoom reference window in a write zoom system of two zoom systems (read zoom and write zoom) employed in an ultrasound medical apparatus, to increase a diagnosis efficiency, and an ultrasound medical apparatus employing the image zoom method.

### SUMMARY

An ultrasound medical apparatus according to some embodiments includes a transducer configured to transmit an ultrasound to a zoom area of a subject based on a write zoom instruction and to receive a first reflected signal corresponding to the ultrasound from the zoom area, a scan converting unit configured to convert the first reflected signal into zoom image data for displaying the first reflected signal and to allow the zoom image data to be displayed in a first window area on a display unit, and a zoom processing unit configured to control the transducer to transmit a plane wave to the subject in a predetermined cycle, to convert a second reflected signal received from the subject into entire image data, and to allow the entire image data to be displayed in a second window area on the display unit.

A method of zooming an image by an ultrasound medical apparatus, according to some embodiments, includes a receiving step including transmitting an ultrasound to a zoom area of a subject based on a write zoom instruction and receiving a first reflected signal corresponding to the ultrasound from the zoom area; a scanning step including converting the first reflected signal into zoom image data for displaying the first reflected signal and allowing the zoom image data to be displayed in a first window area on a display unit, and a zoom processing step including transmitting a plane wave to the subject in a predetermined cycle, converting a second reflected signal received from the subject into entire image data, and allowing the entire image data to be displayed in a second window area on the display unit.

### Advantageous Effects

As described above, according to some embodiments, an entire image is updated in real time in a zoom reference window in a write zoom system of two zoom systems (read zoom and write zoom) employed in an ultrasound medical apparatus, thus increasing a diagnosis efficiency. That is, according to some embodiments of the present disclosure, the entire image is updated in real time in the zoom reference window of the write zoom by using a software-based high-speed image processing.

Further, according to some embodiments of the present disclosure, not only the diagnosis efficiency of the relevant equipment can be increased by way of the real time image update in the zoom reference window, which has not been supported in the write zoom, but also the current scan position of a subject (target to be diagnosed) can be easily located and the diagnosis time can be shortened. That is, the inherent disability of the typical write zoom to update the entire image of the subject in real time in the zoom reference window is overcome by some embodiments, which can update the entire image of the subject in real time together with the zoom image by using a plane wave when employing the write zoom. Hence, according to some embodiments of the present disclosure, when a user wants to see other site while viewing a zoom image corresponding to a zoom area, the viewer can see the entire image that is updated in real time.

### BRIEF DESCRIPTION OF DRAWINGS

FIG. 1 is a block diagram of an ultrasound medical apparatus for zooming an image according to some embodiments of the present disclosure.

FIG. 2 is a flowchart of an image zoom method according to some embodiments of the present disclosure.

FIG. 3 is a schematic diagram for illustrating a read zoom and a write zoom according to some embodiments of the present disclosure.

FIG. 4 is a schematic diagram for illustrating an image processing by using an ultrasound according to some embodiments of the present disclosure.

FIG. 5 is a schematic diagram for illustrating an image processing by using a plane wave according to some embodiments of the present disclosure.

FIG. 6 is a schematic diagram for illustrating an image processing by using an ultrasound and a plane wave according to some embodiments of the present disclosure.

REFERENCE NUMERALS

| | |
|---|---|
| 100: Ultrasonic Medical Apparatus | 110: Transducer |
| 120: Transmission/Reception Switch | 132: Transmitting Unit |
| 134: Receiving Unit | 140: Beamformer |
| 150: Analog-to-Digital Converter | 170: Signal Processing Unit |
| 182: Scan Converting Unit | 184: Zoom Processing Unit |
| 190: Display Unit | |

### DETAILED DESCRIPTION

Exemplary embodiments of the present disclosure are described in detail below with reference to the accompanying drawings.

Ultrasound image data (zoom image data and entire image data) described in some embodiments includes a B-mode image and a C-mode image. The B-mode image is a grayscale image in an image mode for displaying a motion of a target object, and the C-mode image is an image in a color flow image mode. A BC-mode image is an image in an image mode for displaying a blood flow or a motion of the target object by using a Doppler effect, which simultaneously provides the B-mode image and the C-mode image to provide anatomic information together with the blood flow and the motion of the target object. That is, the B-mode is a grayscale image mode for displaying the motion of the target object, and the C-mode is a color flow image mode for displaying the blood flow or the motion of the target object. An ultrasound medical apparatus 100 according to some embodiments is capable of simultaneously providing a B-mode image and the C-mode image that is a color flow image.

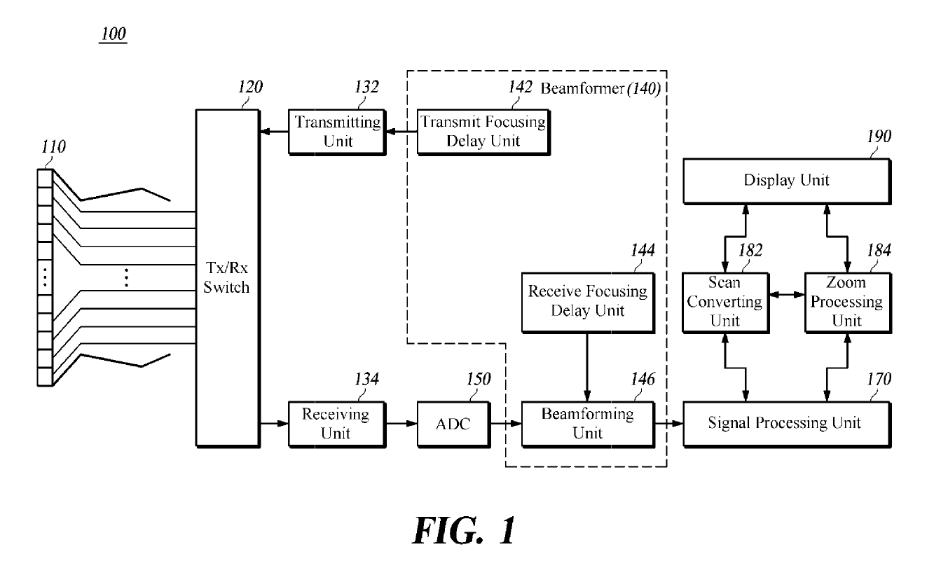

FIG. 1 is a block diagram of an ultrasound medical apparatus for zooming an image according to some embodiments.

The ultrasound medical apparatus 100 according to some embodiments includes a transducer 110, a transmission/reception switch 120, a transmitting unit 132, a receiving unit 134, a transmit focusing delay unit 142, a receive focusing delay unit 144, a beamforming unit 146, an analog-to-digital converter 150, a signal processing unit 170, a scan converting unit 182, a zoom processing unit 184, and a display unit 190. Although it is described that, in some embodiments, the ultrasound medical apparatus 100 includes the transducer 110, the transmission/reception switch 120, the transmitting unit 132, the receiving unit 134, the transmit focusing delay unit 142, the receive focusing delay unit 144, the beamforming unit 146, the analog-to-digital converter 150, the signal processing unit 170, the scan converting unit 182, the zoom processing unit 184, and the display unit 190, this is a mere example instantiating the technical idea of some embodiments, and accordingly, one of ordinary skill in the pertinent art would appreciate that various modifications, additions and substitutions are possible in the constituent elements of the ultrasound medical apparatus 100 without departing from the idea and scope of the embodiments.

The transducer 110 converts an electrical analog signal into an ultrasound, transmits the ultrasound to a subject, receives a signal reflected at the subject (hereinafter, a “reflected signal”), and converts the reflected signal into an electrical analog signal. In general, the transducer 110 includes a plurality of transducer elements coupled to each other. The transducer 110 converts an acoustic energy into an electrical signal, and vice versa. In some embodiments, the transducer 110 includes a transducer array, transmits the ultrasound to the subject by using transducer elements in the transducer array, and receives a reflected signal from the subject.

The transducer 110 includes a plurality of (e.g., 128) transducer elements, and outputs the ultrasound in response to a voltage applied from the transmitting unit 132. At this time, a part of the transducer elements among the plurality of transducer elements is utilized for the transmission of the ultrasound. For example, at the time of transmitting the ultrasound, even when the transducer 110 includes 128 transducer elements, only 64 transducer elements can transmit the ultrasound to form a transmit scanline. The transducer 110 can be used for both transmission and reception.

The transducer 110 transmits the ultrasound to a zoom area selected by a user in a region of interest (ROI) in order to perform a write zoom, and receives a first reflected signal corresponding to the ultrasound from the zoom area, or transmits a plane wave to the subject and receives a second reflected signal corresponding to the reflected plane wave from the subject. The transducer 110 can be implemented with 1D (Dimension), 1.25D, 1.5D, 1.75D or 2D transducer array. For example, when the transducer 110 is implemented with 1 D, 1.25D, 1.5D and 1.75D, the transducer 110 transmits the ultrasound to the zoom area by rotation through predetermined angles (0 degrees to 360 degrees) and then receives the first reflected signal corresponding to the ultrasound from the zoom area, or transmits the plane wave to the subject and then receives the second reflected signal corresponding to the plane wave from the subject. When the transducer 110 is implemented with 2D, the transducer 110 transmits the ultrasound to the zoom area without performing the rotation and then receives, from the zoom area, the first reflected signal corresponding to the ultrasound, or transmits the plane wave to the subject and then receives the second reflected signal corresponding to the reflected plane wave from the subject.

The transducer 110 transmits an ultrasound beam focused by appropriately delaying the input times of pulses to the respective transducer elements, to the subject along the transmit scanline. The first reflected signal from the zoom area and the second reflected signal from the subject are inputted to the transducer 110 at different reception times, respectively, and the transducer 110 delivers the inputted first reflected signal or second reflected signal to a beamformer 140.

The transducer 110 according to some embodiments transmits the ultrasound to the zoom area of the subject based on the write zoom instruction, and receives the first reflected signal corresponding to the ultrasound from the zoom area. That is, when a user wants to zoom in an ultrasound image outputted to the display unit 190, he or she inputs a zoom instruction by way of a user input unit. At this time, the zoom instruction includes either one of a read zoom instruction and a write zoom instruction. Thereafter, the user selects a zoom area to be zoomed in from the ultrasound image outputted to the display unit 190 by using a cursor or the like. Further, the transducer 110 according to some embodiments transmits the plane wave to the subject based on the write zoom instruction, and receives the second reflected signal corresponding to the plane wave from the subject. The transducer 110 transmits the ultrasound to the zoom area along a predetermined scanline and then receives the first reflected signal from the zoom area, or transmits the plane wave to the subject by using an entire predetermined scanline and then receives the second reflected signal from the subject.

The transmission/reception switch 120 performs a function of switching between the transmitting unit 132 and the receiving unit 134, such that the transducer 110 performs transmission and reception in an alternate manner. Further, the transmission/reception switch 120 takes a role of preventing a voltage outputted from the transmitting unit 132 from affecting the receiving unit 134.

The transmitting unit 132 applies a voltage pulse to the transducer 110, to cause each of the transducer elements of the transducer 110 to output the ultrasound. The receiving unit 134 receives the reflected signal (first reflected signal and second reflected signal), which is the ultrasound outputted from each of the transducer elements of the transducer 110 and reflected at the subject. The receiving unit 134 then processes the reflected signal (first reflected signal and second reflected signal) through an amplification, a removal of aliasing phenomenon and noise component, a compensation for an attenuation generated while the ultrasound signal passes through the inside of a body, and the like, to obtain a post-processed signal, and transmits the post-processed signal to the analog-to-digital converter 150.

The beamformer 140 appropriately delays electrical signals for the transducer 110, to convert the electrical signals into an electrical signal suitable for each of the transducer elements. The beamformer 140 delays or sums electrical signals respectively converted by the transducer elements, to calculate an output value of a corresponding transducer element. The beamformer 140 includes a transmit beamformer, a receive beamformer and a beamforming unit 146. The transmit beamformer corresponds to the transmit focusing delay unit 142, and the receive beamformer corresponds to the receive focusing delay unit 144. The beamformer 140 according to some embodiments generates a first delay time required to focus the ultrasound on the zoom area or generates a second delay time required to focus the plane wave on the subject, and then generates a combined signal by combining digital signals to which the first delay time or the second delay time is applied. In some embodiments, the beamformer 140 is connected to the signal processing unit 170 via a full parallel path, in order to perform a software-based high-speed image processing.

The transmit focusing delay unit 142 adds an appropriate delay to each electrical digital signal by considering time to reach each of the transducer elements from the subject (to be diagnosed). That is, when the transducer 110 includes a transducer array, the transmit focusing delay unit 142 adjusts the beam and electronically focuses the beam. The transducer array is supposed to electronically focus the beam according to different depths, while the transmit focusing delay unit 142 focuses the beam on the transmission side by successively applying a pulse delay time to each of the transducer elements of the transducer array. Consequently, the transmit focusing delay unit 142 adjusts the direction of the beam with respective to the transducer array that is electronically scanned.

The receive focusing delay unit 144 generates a delay time required to focus the digital signal converted by the analog-to-digital converter 150 or to perform a beamforming. That is, the receive focusing delay unit 144 provides a delay time for focusing the reflected signal received from the transducer 110 and adjusts a dynamic focusing of the reflected signal.

The beamforming unit 146 forms a receive focusing signal by summing the electrical digital signals converted by the analog-to-digital converter 150. The beamforming unit 146 combines the digitalized signals into a single signal. At this time, reflected signals having the same phase are coupled by the beamforming unit 146, and after being subjected to various signal processing methods by the signal processing unit 170, outputted by the display unit 190 via the scan converting unit 182. The beamforming unit 146 applies different amounts of delay (that depend on where the receive focusing is desired) to the signals received from the analog-to-digital converter 150, and performs the dynamic focusing by combining delayed signals. That is, the beamforming unit 146 combines the reflected signals respectively received from the transducer elements into a single signal for a subsequent signal processing. The beamforming unit 146 generates a combined signal obtained by combining the reflected signals received from all the transducer elements, in order to form a single reflected signal for each reflecting member (subject). The combined signal generated in this manner is transmitted from the beamforming unit 146 to the signal processing unit 170, and finally transmitted to a digitalizing device that converts the combined signal into a digital signal for image data storage.

The analog-to-digital converter 150 converts the analog reflected signal into a digital signal and then transmits the digital signal to the beamforming unit 146. The reflected signal as received by the analog-to-digital converter 150 from the transducer 110 takes the form of analogue signal that is represented by a continuous voltage signal. At this time, the analog signal needs to be converted to a digital signal before it is processed by the scan converting unit 182. And therefore, the analog-to-digital converter 150 converts the analog form of reflected signal into a combination of 0s and 1s. The digitized binary signal from the analog-to-digital converter 150 goes through the signal processing unit 170 and is stored in the memory of the scan converting unit 182. The analog-to-digital converter 150 according to some embodiments converts the first reflected signal or a second reflected signal into a digital signal.

The signal processing unit 170 converts the reflected signals of receive scanlines, focused by the beamforming unit 146 to baseband signals, detects an envelope by using a quadrature demodulator to obtain data for a single scanline. Furthermore, signal processing unit 170 performs the A/D conversion of the data generated by the beamformer 140 into digital signal.

For the purpose of a fast imaging of the second reflected signal corresponding to the plane wave, the signal processing unit 170 may perform a software-based parallel processing of the relevant data. Specifically, the signal processing unit 170 compares the input data sequence with a comparison data string; generates a comparison result data string; extracts a representative bit from each of the comparison result data that make up the comparison result data string; generates a representative bit string based on representative bits; saves, in a table, a plurality of operation data sequences corresponding to a bit pattern that can be represented by the representative bit string; utilizes a particular operation data sequence from the plurality of operation data sequences, which is selected according to the representative bit strings; and perform a data operation of the input data sequence so as to generate a radiation quantity data sequence. The signal processing unit 170 performs the software-based parallel processing for high-speed imaging processing, while its architectural equivalent may have a multi-core CPU (Central Processing Unit) and a GPU (Graphic Processing Unit) for carrying out the parallel processing in thousands of channels at the same time.

The scan converting unit 182 records the data obtained by the signal processing unit 170 into memory, directs the data scanning to match the pixel direction of the display unit 190 (i.e., monitor), and maps the corresponding data to the pixel positions on the display unit 190. The scan converting unit 182 converts the ultrasound image data (zoom image data, entire image data) to a data format for use in the display unit 190 having a predetermined scanline display format.

The primary role of the scan converting unit 182 is to store temporary ultrasound image data (zoom image data, entire image data). The scan converting unit 182 receives a reflected signal from the transducer 110, and then stores a reflected signal received in the internal memory (i.e., storage device). Then, the scan converting unit 182 converts the reflected signal into image data and outputs the same on the display unit 190. In this case, image data may be converted into B-mode image data as well as M-mode image data, Doppler mode image data and color flow mode image data. If the scan converting unit 182 is not in the stop mode, the reflected signal stored in the internal memory is constantly updated to be new information. Here, the converted image data is output to the display unit 190 as the same data is updated in real time. On the other hand, in the stop mode, the scanning operation is stopped, and the scan converting unit 182 performs only the output function. The scan conversion of the scan converting unit 182 is required because the format of the image acquisition is different from that of its reconstruction, and the ultrasound image data is output on the display unit 190. In this case, the reflected signals reach the scan converting unit 182 along the respective scanlines. In addition, the memory of the scan converting unit 182 takes a buffer role between different data formats while writing and reading the data. The scan converting unit 182 receives the reflected signal at the information format and the speed of the transducer 110. The scan converting unit 182 records the reflected signal as a unit of image data in the memory. The image data is read from the memory by the scan converting unit 182 for display on the display unit 190 or monitor and is processed to conform to the horizontal image scanning by the display unit 190.

The memory of the scan converting unit 182 can be recognized as a matrix of elements each made up of multi-bit storage units with respect to the ultrasound image data received from a preset position. Here, the digitized element is referred to as of a pixel. That is, the memory of the scan converting unit 182 is a matrix of such pixels. Ultrasound image data that is output on the display unit 190 is actually present in the memory of the scan converting unit 182, in a matrix form of a digital number. During a probing operation, the reflected signal is inserted in the pixel position (address) depending on the location of the object. In order to calculate the exact pixel addresses, the scan converting unit 182 uses the delay time of the reflected signal and beam coordinates of the transducer 110.

At this time, to present the value of the reflected signal on the position of each pixel, the scan converting unit 182 operates on at least eight bits. An 8-bit has 256 amplitude levels at each position. Such memory of the scan converting unit 182 is constantly updated with new reflected signal information as the ultrasonic beam proceeds to the ROI. Meanwhile, the image stop function of the scan converting unit 182 enables the reflected signal to be stored in the memory for not only image recording but also photo or other digital information storage. Memory of the scan converting unit 182 provides its output by transmitting the values of the pixels to the digital-analog converter (DAC) for supplying the necessary signals to adjust the level of brightness of the display unit 190.

The scan converting unit 182 according to an embodiment converts the first reflected signal into a zoom image data for displaying on the display, and renders the zoom image data to be displayed at a first window area on the display unit 190. Here, the first window area refers to an image window area that is a main section.

The zoom processing unit 184 according to an embodiment operates the transducer 110 to transmit the plane wave to the subject at a predetermined cycle, convert the second reflected signal from the subject into entire image data to be displayed, and render the entire image data so to be presented at a second window area of the display unit 190. The second window area refers to a zoom reference window area.

The zoom processing unit 184 operates to render the zoom image data on the main image window area, while simultaneously rendering the entire image data on the zoom reference window area that is a sub-section. At this time, the zoom reference window area is included in the image window area. The zoom processing unit 184 performs a real-time updating of the entire image data which has been generated based on the second reflected signal, on the zoom reference window area. At this time, the second reflected signal is a signal corresponding to the plane wave and it may undergo a software-based high-speed imaging process. The zoom processing unit 184 controls the transducer 110 so as to transmit the plane wave to the subject based on the pre-set time or pre-set frame. That is, the zoom processing unit 184 operates the transducer 110 so as to transmit the plane wave to the subject in unit of predetermined seconds. For example, the transmission cycle of the plane wave may be set by one of seconds, milliseconds and microseconds, and the zoom processing unit 184 may transmit the plane wave to the subject by seconds. Moreover, the zoom processing unit 184 operates the transducer 110 to transmit, to the object the plane wave by a number of times predetermined for frames of a certain number determined with respect to preset frames. For example, the transmission cycle of the plane wave may be set to one plane wave per frame, and the zoom processing unit 184 may accordingly transmit a plane wave to the subject once per frame.

The zoom processing unit 184 operates the transducer 110 to transmit the plane waves at a plurality of angles to the subject, receive the resultant second reflected signals respectively, and then generate an entire image data obtained by synthesizing the second reflected signals. The zoom processing unit 184 may control the transducer 110 to transmit the plane wave once to the subject, or to transmit the same a number of times. When the zoom processing unit 184 has the transducer 110 transmit the plane wave multiple times, the transducer 110 may transmit the plane wave at different angles to the subject, receive the corresponding second reflected signals respectively, and then generate the entire image data by synthesizing the second reflected signals. The second reflected signal corresponds to the plane wave, and therefore it can readily enter the software-based high-speed imaging process. Moreover, the zoom processing unit 184 may operate the transducer 110 to consecutively transmit ultrasonic waves to the zoom area in response to an input zoom instruction, and to pause the ultrasonic transmission in every predetermined cycle, and instead transmit the plane wave to the subject.

In some embodiments, the ultrasound medical apparatus 100 further includes a user input unit which receives an instruction from an operation or an input of a user. In some embodiments, the user instruction includes a setting instruction for controlling the ultrasound medical apparatus 100 and the like.

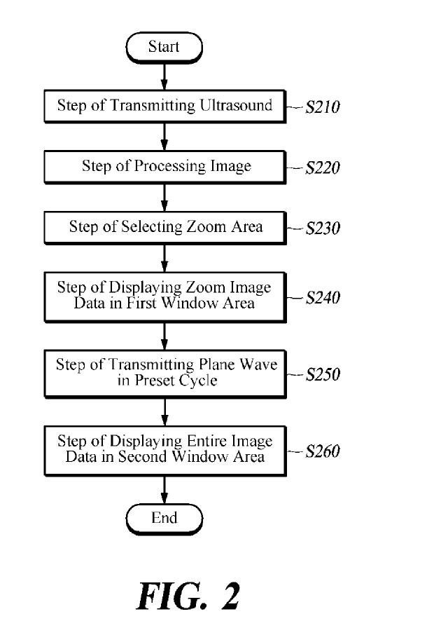

FIG. 2 is a flowchart of an image zoom method according to some embodiments.

The ultrasound medical apparatus 100 transmits the ultrasound to the subject, and receives the first reflected signal corresponding to the ultrasound from the subject (step S210). The ultrasound medical apparatus 100 converts the first reflected signal into ultrasound image data, and outputs the ultrasound image data via the display unit 190 (step S220). When a zoom instruction is inputted by an operation or an instruction from a user, the ultrasound medical apparatus 100 selects a zoom area for zooming in from the ultrasound image data based on the zoom instruction (step S230). In step S230, when a user wants to zoom in the ultrasound image displayed on the display unit 190, he or she inputs a zoom instruction by way of the user input unit. At this time, the zoom instruction can be selected as either one of a write zoom instruction and a read zoom instruction. Thereafter, the user selects the zoom area to be zoomed in from the ultrasound image outputted to the display unit 190 by using a “cursor” or the like.

After step S230, The ultrasound medical apparatus 100 transmits the ultrasound to the zoom area based on the write zoom instruction, and receives the first reflected signal corresponding to the ultrasound from the zoom area. The ultrasound medical apparatus 100 converts the first reflected signal for the zoom area into zoom image data for displaying the first reflected signal, and allows the zoom image data to be displayed in the first window area on the display unit 190 (step S240). The first window area is the image window area, which is the main area. In step S240, the ultrasound medical apparatus 100 transmits the ultrasound to the zoom area along a predetermined scanline, and then receives the first reflected signal from the zoom area.

The ultrasound medical apparatus 100 transmits the plane wave to the subject in a predetermined cycle (step S250). In step S250, the ultrasound medical apparatus 100 allows the plane wave to be transmitted to the subject based on a predetermined time or a predetermined frame. That is, the ultrasound medical apparatus 100 allows the plane wave to be transmitted to the subject based on a predetermined unit of second. For example, the transmission cycle of the plane wave can be set any one of units of second, millisecond, microsecond, and nanosecond, and the ultrasound medical apparatus 100 can transmit the plane wave based on the predetermined unit of second. Further, the ultrasound medical apparatus 100 allows the plane wave to be transmitted to the subject for predetermined times per frame based the predetermined frame. For example, the transmission cycle of the plane wave can be set as a single time per frame, and the ultrasound medical apparatus 100 can transmit the plane wave once for every frame.

The ultrasound medical apparatus 100 converts the second reflected signal into the entire image data, allows the entire image data to be displayed onto the second window area of the display unit 190 (Step S260). The second window area refers to the zoom reference window area. In Step S260, the ultrasound medical apparatus 100 transmits the plane wave to the subject by using a predetermined entire scanline and receives the second reflected signal from the subject. In addition, the ultrasound medical apparatus 100 displays the zoom image data in the image window area (the first window area) which is a main area, and at the same time, displays the entire image data in the zoom reference window area (the second window area) which is an auxiliary area (sub area). The zoom reference window area (the second area) is included in the image window area (the first area).

In step S260, the ultrasound medical apparatus 100 transmits the plane wave at a plurality of angles to the subject, receives the second reflected signals corresponding to the angles, and generates the entire image data by combining the second reflected signals. In some embodiments the ultrasound medical apparatus 100 transmit the plane wave once. In some embodiments, the ultrasound medical apparatus 100 transmits the plane multiple times. In case of the multiple transmissions, the ultrasound medical apparatus 100 transmits the plane wave at a plurality of angles to the subject, receives the second reflected signals corresponding to the angles, and generates the entire image data by combining the second reflected signals. Thereafter, the ultrasound medical apparatus 100 performs a real-time update of the entire image data which is based on the second reflected signals and displays the update image data in the zoom reference window area. The second reflected signal is the signal that corresponds to the plane wave, and in some embodiments, it is subjected to a software-based high-speed image processing.

In steps S250 and S260, the ultrasound medical apparatus 100 continuously transmits the ultrasound wave to the zoom area upon receiving a zoom instruction. It transmits the plane wave instead of the ultrasound wave at the end of a predetermined period.

Although steps S210 to S260 are described to be sequentially performed in the example shown in FIG. 2, they merely instantiate a technical idea of the third embodiment. Therefore, a person having ordinary skill in the pertinent art could appreciate that various modifications, additions, and substitutions are possible by changing the sequences described in FIG. 2 or by executing two or more steps from S210 to S260 in parallel, without departing from the gist and nature of the third embodiment, and hence FIG. 2 is not limited to the illustrated chronological sequences.

The image zoom method according to the embodiment shown in FIG. 2 can be implemented as a computer program, and can be recorded on a computer-readable medium. The computer-readable recording medium on which the image zoom method according to the embodiment is recordable includes any type of recording device on which data that can be read by a computer system are recordable. Examples of the computer-readable recording medium include a ROM, a RAM, a CD-ROM, a magnetic tape, a floppy disk, an optical data storage device, and the like, and also include one implemented in the form of carrier wave (e.g., transmission through the Internet). Further, the computer-readable recording medium can be distributed in computer systems connected via a network, and computer-readable codes can be stored and executed in a distributed mode. Moreover, functional programs, codes, and code segments for implementing the third embodiment can be easily deduced by a programmer in technical fields to which the third embodiment belongs.

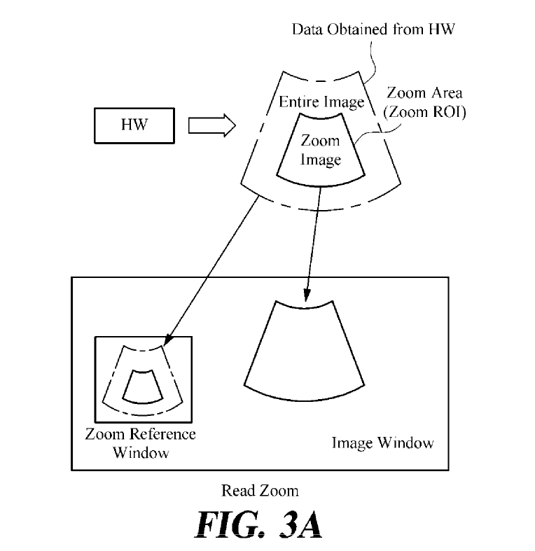

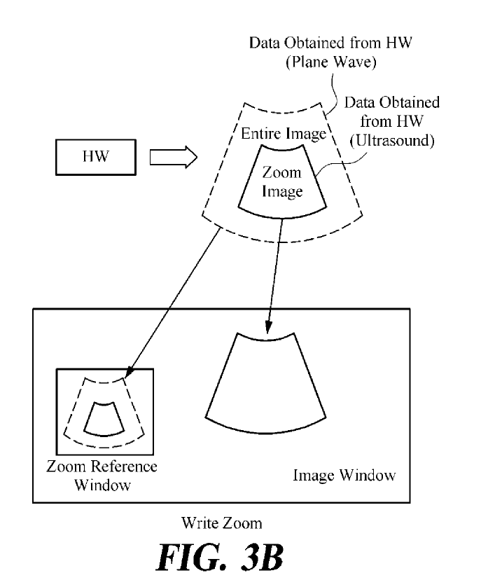

FIG. 3 is a schematic diagram for illustrating a read zoom and a write zoom according to some embodiments.

The image zoom techniques employed by the ultrasound medical apparatus 100 in accordance with the embodiments of the disclosure are so-called a read zoom and a write zoom. The ultrasound medical apparatus 100 supports the functions of can zoom-in and/or zoom-out of the region of interest (ROI) and accordingly can zoom-in and/or zoom-out the user-selected area (zoom area). The ultrasound medical apparatus 100 is capable of zooming-in and/or zooming-out images by either the read zoom or the write zoom scheme. The standard image of the subject under diagnosis (patient) can be obtained by scanning the entire ROI and this image can be zoomed-in upon the user's choice of the area of interest.

The “read zoom” shown in FIG. 3A is a technique that zooms in a specific area (zoom area) in the state that the displayed image freezes. On the other hand, the “write zoom” fill the screen or a specific area of the screen with the data of a specific area (zoom area) which is a part of the data of a frame which is stored in the memory of the scan converting unit 182. As for the “read zoom”, the pixel values corresponding to the empty pixels due to the gap between the before-zoom data and the pixels of the screen can be obtained by using a linear interpolation method. This inevitably exacts a degradation of the quality of the zoom image. Accordingly, the brightness of the zoomed image may become different from that of the original, a moire effect may occur, and a blocking effect may also occur in the zoomed image, which leads to a degradation of an image quality of a particular area. Again, the “read zoom” displays an image with existing limited data to fill the screen or a part of the screen. The existing limited data of the ROI is processed to fill the entire display unit 190. Though this technique provides an advantage that the patient may not be scanned every time the zoom operation is performed since this can be done at the freeze stat. It has, however, a disadvantage that it usually fails to provide a high-definition zoomed image. The “read zoom” can be performed at the post-processing stage of the scan converting unit 182.

On the other hand, the “write zoom” as shown in FIG. 3B, is a technique that the user selects an area to be enlarged (zoom area) by using e.g., a cursor on the original image. Once zoom area is selected, the transducer 110 transmits again to the area to be zoomed. Only the data on the reflected signal corresponding to the area to be zoomed is recorded into the memory of the scan converting unit 182 and these data or all the pixels of the memory are used to fill the screen. The ultrasound medical apparatus 100 can have the option of choosing either of the techniques when it displays the zoomed image. As described above, the “read zoom” technique can display the zoomed image using an existing data without further scanning, while the “write zoom” technique is required to re-scan the to-be-zoomed area. The write zoom technique should obtain the real-time scan image data instead of re-using an existing data. Therefore the “write zoom” can be performed at the pre-processing stage of the scan converting unit 182.

In order to keep or enhance the quality of the image of the zoom ROI, the “write zoom” obtains image data that corresponds only to the ROI. According to this technique, the data corresponding to the area except the zoomed area or the ROI of the zoom reference window is left un-updated and maintains itself as of the zoom operation.

Herewith, the “pre-processing” and the “post-processing can be explained as follows. The former one is a signal process that occurs before the reflected signal is recorded in the memory of the scan converting unit 182, and the latter one is a signal process that occurs after the reflected signal is recorded in the memory of the scan converting unit 182. The former one again can be regarded as a selection of another type of signal compression emphasizing a reflected signal within a specific magnitude range. Further, the signal corresponding to each pixel position can be combined with previous signal of the same position that has been obtained from previous scans. In contrast, the latter can display the stored reflected signal with a variety of brightness levels on the display unit 190, when given options for a variety of adjustments. Therefore, the “post-process” also means a manipulation of the stored data. While the “pre-processing” can be applied to the to-be-stored data, the “post-processing” can be applied to display the existing stored data.

The ultrasound medical apparatus 100 according to some embodiments displays the zoom image data on the “image window” shown in FIG. 3B and at the same time, displays the entire image data on the “zoom reference window”. The zoom image data displayed on the “zoom reference window” is updated in real time. That is, the zoom image data displayed on the “image window” is an image formed based on the ultrasound by a hardware (i.e., transducer 110) and the entire image data is also formed based on the plane wave by the hardware (i.e., transducer 110).

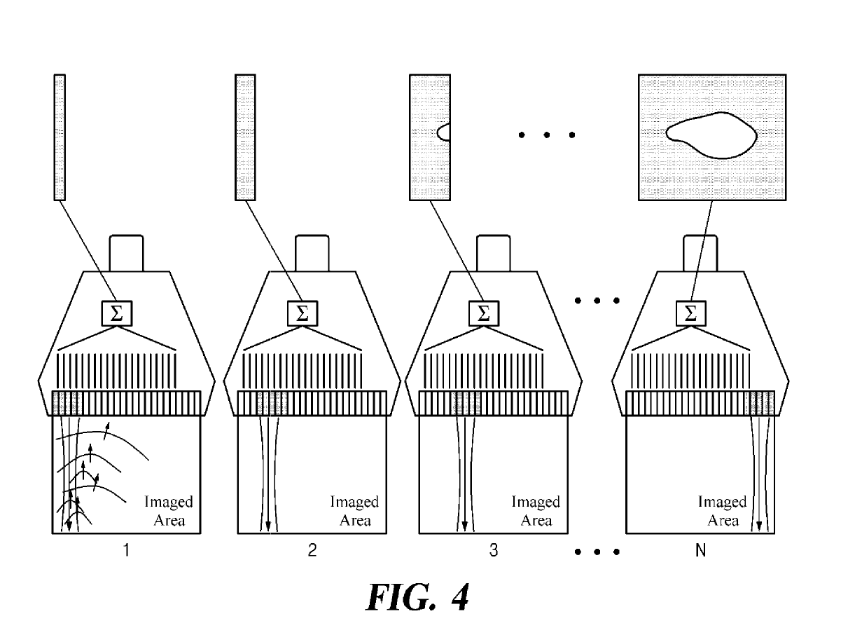

FIG. 4 is a schematic diagram for illustrating an image processing by using an ultrasound according to some embodiments.

As shown in FIG. 4, the image synthesis method performed by the ultrasound medical apparatus 100 is carried out in the manner that one ultrasound beam is used for one scanline of the image. More specifically, the ultrasound medical apparatus 100 transmits the ultrasound signal to the zoom area along a predetermined scanline and receives the first reflected signal from the zoom area. It then converts the first reflected signal by scanline into the ultrasound image data and displays the converted data onto the display unit 190. For example, in case there are scanlines from the first scanline through the N-th scanline, the ultrasound medical apparatus 100 transmits the ultrasound signal along the first scan and performs image processing upon receiving the first reflected signal, and the same process is repeatedly carried out with respect to the second to the N-th scanlines, in order to yield the final image.

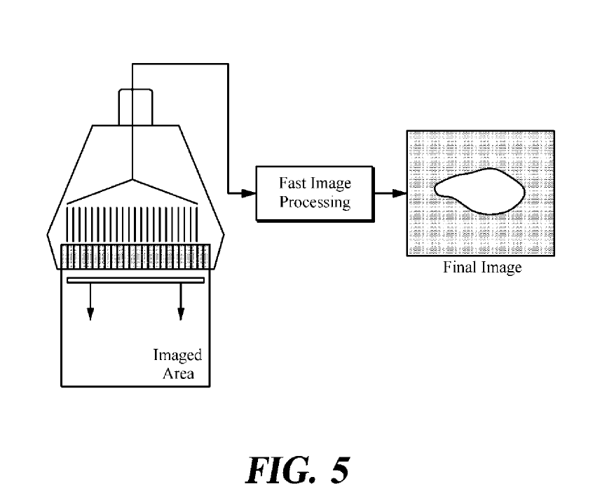

FIG. 5 is a schematic diagram for illustrating an image processing by using a plane wave according to some embodiments.

As shown in FIG. 5, the image processed by the ultrasound medical apparatus 100 by generating the plane wave is fast obtained as compared with conventional methods because all the transducer elements are involved at a time to produce the final image. Specifically, the ultrasound medical apparatus 100 transmits the plane wave to the subject, converts the second reflected signal corresponding to the plane wave into the entire image data, and finally exhibits the entire image data on the display unit 190. When converting the second reflected signal into the entire image data, the ultrasound medical apparatus 100 may executes a software-based parallel processing for the fast image processing.

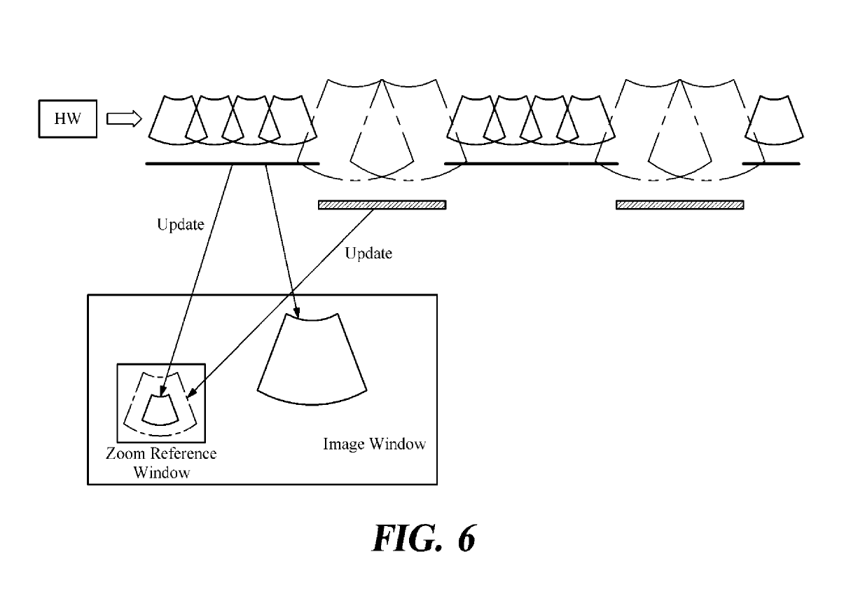

FIG. 6 is a schematic diagram for illustrating an image processing by using an ultrasound and a plane wave according to some embodiments.

As shown in FIG. 6, upon receiving the write zoom instruction, the ultrasound medical apparatus 100 transmits the ultrasound signal to the zoom area of the subject, receives the first reflected signal from the zoom area, converts the first reflected signal into the zoom image data, and exhibits the zoom image data in the image window area (the first window area) on the display unit 190. In the meantime, while it exhibits the zoom image data in the image window area (the first window area), the ultrasound medical apparatus 100 transmits the plane wave to the subject with a predetermined period, converts the second reflected signal which is reflected from the subject into the entire image data, and exhibits the entire image data in the zoom reference window area (the second window area) on the display unit 190.

The ultrasound medical apparatus 100, as of the “write zoom”, performs a real-time update/renew with respect to the zoom reference window (the second window), after obtaining the entire image data finally obtained by transmitting the periodic plane wave. Consequently, the ultrasound medical apparatus 100 can perform relatively fast update/renew of the real-time image data displayed in the zoom reference image window (the second window) without regard to the FPS (frame per second). This is possible because it can converts with a very high speed the second reflected signal into the entire image data and because the second reflected data is made from the plane wave which is formed by the software-based beamforming.

In the meantime, the ultrasound medical apparatus 100 transmits the plane wave by a predetermined time or by a predetermined frame, repeatedly transmits the ultrasound wave to the zoom area upon receiving the write zoom instruction. It transmits the plane wave to the subject instead of transmitting the ultrasound wave at each end of a predetermined period.

Although exemplary embodiments have been described for illustrative purposes, those skilled in the art will appreciate that various modifications, additions and substitutions are possible, without departing from the idea and scope of the claimed disclosure. Accordingly, one of ordinary skill would understand the scope of the claimed disclosure is not to be limited by the explicitly described above embodiments but by the claims and equivalents thereof.

### CROSS-REFERENCE TO RELATED APPLICATION

If applicable, this application claims priority under 35 U.S.C §119(a) of Patent Application No. 10-2013-0048827, filed on Apr. 30, 2013 in Korea, the entire content of which is incorporated herein by reference. In addition, this non-provisional application claims priority in countries, other than the U.S., with the same reason based on the Korean patent application, the entire content of which is hereby incorporated by reference.

1. An ultrasound medical apparatus, comprising:
a transducer configured
to transmit an ultrasound to a zoom area of a subject based on a write zoom instruction, and
to receive a first reflected signal corresponding to the ultrasound from the zoom area;
a scan converting unit configured
to convert the first reflected signal into zoom image data for displaying the first reflected signal, and
to allow the zoom image data to be displayed in a first window area on a display unit; and
a zoom processing unit configured
to control the transducer to transmit a plane wave to the subject in a predetermined cycle,
to convert a second reflected signal received from the subject into entire image data, and
to allow the entire image data to be displayed in a second window area on the display unit.

2. The ultrasound medical apparatus according to claim 1, wherein the zoom processing unit is configured
to allow the zoom image data to be displayed in an image window area that is a main area, and
to allow the entire image data to be displayed in a zoom reference window that is a sub area.

3. The ultrasound medical apparatus according to claim 2, wherein the zoom processing unit is configured to allow the entire image data to be updated in real time in the zoom reference window.

4. The ultrasound medical apparatus according to claim 2, wherein the zoom reference window is included in the image window area.

5. The ultrasound medical apparatus according to claim 1, wherein the zoom processing unit is configured to control the transducer to transmit the plane wave to the subject based on a predetermined time or a predetermined frame.

6. The ultrasound medical apparatus according to claim 5, wherein the zoom processing unit is configured to control the transducer to transmit the plane wave to the subject based on a predetermined unit of second.

7. The ultrasound medical apparatus according to claim 5, wherein the zoom processing unit is configured to control the transducer to transmit the plane wave to the subject for predetermined times per frame based the predetermined frame.

8. The ultrasound medical apparatus according to claim 1, wherein the transducer is configured
to transmit the ultrasound to the zoom area along a predetermined scanline and then receive the first reflected signal from the zoom area, or
to transmit the plane wave to the subject and then receive the second reflected signal from the subject.

9. The ultrasound medical apparatus according to claim 1, further comprising:
an analog-to-digital converter (ADC) configured to convert the first reflected signal or the second reflected signal into a digital signal; and
a beamformer configured
to generate a first delay time required to focus the ultrasound on the zoom area,
to generate a second delay time required to focus the plane wave on the subject, and
to generate a combined signal by combining digital signals on which the first delay time or the second delay time is applied.

10. The ultrasound medical apparatus according to claim 1, wherein the zoom processing unit is configured
to control the transducer to transmit the plane wave to the subject at a plurality of angles and to receive second reflected signals respectively corresponding to the angles, and
to generate the entire image data by combining the second reflected signals.

11. The ultrasound medical apparatus according to claim 1, wherein the zoom processing unit is configured to control the transducer
to transmit the ultrasound to the zoom area continuously based on the write zoom instruction, and
to pause transmission of the ultrasound and transmit the plane wave to the subject in a predetermined cycle.

12. A method of zooming an image by an ultrasound medical apparatus, the method comprising:
a receiving step including
transmitting an ultrasound to a zoom area of a subject based on a write zoom instruction, and
receiving a first reflected signal corresponding to the ultrasound from the zoom area;
a scanning step including
converting the first reflected signal into zoom image data for displaying the first reflected signal, and
allowing the zoom image data to be displayed in a first window area on a display unit; and
a zoom processing step including
transmitting a plane wave to the subject in a predetermined cycle,
converting a second reflected signal received from the subject into entire image data, and
allowing the entire image data to be displayed in a second window area on the display unit.

13. The method according to claim 12, wherein the zoom processing step includes
allowing the zoom image data to be displayed in an image window area that is a main area, and
allowing the entire image data to be displayed in a zoom reference window that is a sub area.

14. The method according to claim 12, wherein the zoom processing step includes transmitting the plane wave to the subject based on a predetermined time or a predetermined frame.

15. The method according to claim 12, wherein the zoom processing step includes
transmitting the ultrasound to the zoom area continuously based on the write zoom instruction, and
pausing transmission of the ultrasound and transmitting the plane wave to the subject in a predetermined cycle.
